(19) United States
(12) Patent Application Publication
(10) Pub. No.: US 2025/0116797 A1
(43) Pub. Date: Apr. 10, 2025

(54) METHOD FOR PREPARING ESTER THIOL COMPOUND AND OPTICAL RESIN CONTAINING THE SAME

(71) Applicant: KS OPTICAL CO., LTD., Suncheon-si, Jeollanam-do (KR)

(72) Inventors: Keun Sik KIM, Suncheon-si, Jeollanam-do (KR); Kwang Young LEE, Gwangyang-si, Jeollanam-do (KR)

(21) Appl. No.: 18/832,917

(22) PCT Filed: Jan. 25, 2022
(86) PCT No.: PCT/KR2022/001284

§ 371 Date: Jul. 24, 2024

(51) Int. Cl. G02B 1/04; C07C 323/51; C08G 18/38

(52) U.S. Cl. CPC G02B 1/04; C07C 323/51; C08G 18/3855

(57) ABSTRACT

A method for producing an ester thiol compound of Formula 3 below includes: reacting a polyalcohol of Formula 1 below and a mercaptocarboxylic acid of Formula 2 below by using a catalyst, wherein the catalyst is prepared by introducing superstrong acid into mesoporous materials with pore diameter ranging from 2 to 10 nm:

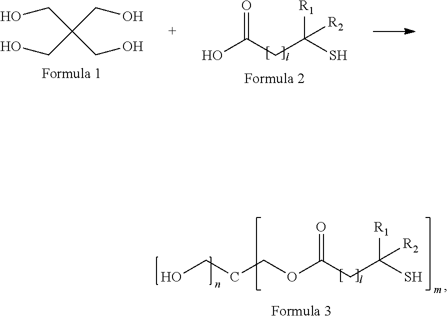

wherein, R₁ and R₂ are each independently H or CH₃, l is an integer of 0 to 2, m is an integer of 1 to 4, and n is an integer of 0 to 3.

### TECHNICAL FIELD

The present disclosure relates to a method for producing an ester thiol compound, and a poly(thio)urethane-based polymerizable composition and optical resin or an optical resin containing the ester thiol compound obtained by the method and having superior optical properties.

### TECHNICAL BACKGROUND

A polythiourethane-based optical material is widely used as various optical materials such as spectacle lenses and camera lenses because it has a similar refractive index to a glass optical material, is lighter and stronger against impact compared to glass optical material, and has excellent dyeing properties. Recently, high functionality such as impact resistance and heat resistance of the optical materials is required, and high optical transparency, high refractive index, and high Abbe number performance are also required.

Research on ester thiol compounds has been steadily attempted to improve the impact resistance of polythiourethane-based optical materials, and a composition for optical materials containing an ester thiol compound can be used as a thio-ene composition for photocuring and a curing agent for an epoxy curing system.

An ester thiol compound has been produced through an esterification method in which an ordinal polyhydric alcohol is reacted with an ordinal mercaptocarboxylic acid in the presence of an esterification catalyst, while distilling off water, which is produced as a by-product (refer to Prior Art Literatures 1 to 4).

However, a conventional method for producing an ester thiol compound uses a homogeneous catalyst, and thus a special purification process is required to remove the homogeneous catalyst after the reaction. Consequently, additional process costs may be occurred and large amounts of wastewater may be generated, which may lead to environmental pollution. In addition, some of the homogeneous catalyst may remain in the ester thiol compound, which may cause striae, white turbidity, yellowing, etc. when producing optical resins.

### PRIOR ART LITERATURE

(1) Korean Patent Application No. 10-2010-7028490
(2) Korean Patent Application No. 10-2016-0090624
(3) Korean Patent Application No. 10-2017-7035596
(4) Korean Patent Application No. 10-2019-0005901

### DETAILED DESCRIPTION

### Problems to be Solved

Therefore, the present inventor has studied to solve the above-mentioned shortcomings, and as a result, found that by introducing a solid acid independent of a reaction system to produce an ester thiol compound, the additional complex purification process derived from the use of a homogeneous catalyst can be converted into a simple process such as filtering to simplify the production process, thereby efficiently producing an ester thiol compound. In addition, it is possible to reuse the used catalyst and thus suppress the amount of wastewater and waste generated, so that an economical and environmentally friendly process can be differentiated from existing processes. Therefore, the present disclosure can reduce process costs and obtain high yield and high quality products, thereby providing economic benefits in terms of industrialization

### SUMMARY OF INVENTION

In order to achieve the above object of the present disclosure, there is a provided a method for producing an ester thiol compound of Formula 3 below, the method comprising reacting a polyalcohol of Formula 1 below and a mercaptocarboxylic acid of Formula 2 below by using a heterogeneous solid acid catalyst:

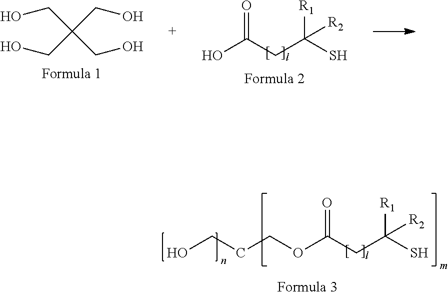

[wherein, R₁ and R₂ are each independently H or CH₃, l is an integer of 0 to 2, m is an integer of 1 to 4, and n is an integer of 0 to 3].

Wherein the solid acid catalyst may be a natural clay, synthetic silica-alumina, zeolite, ion exchange resin, heteropoly acid, or superstrong acid mesoporous material catalyst. The solid acid catalyst is preferably a superstrong acid mesoporous material catalyst. Particularly, a mesoporous material into which sulfonic acid is introduced as a superstrong acid is more preferred.

Meanwhile, the superstrong acid mesoporous material catalyst may be used in a weight ratio of 0.2 to 20, preferably in a weight ratio of 12 to 20.

Further, the polyalcohol of Formula 1 and the mercaptocarboxylic acid of Formula 2 may be used in a ratio of 20˜30 to 80˜70.

Further, the method of the present disclosure is carried out preferably in a fixed bed tube type reactor in which the heterogeneous solid acid catalyst is packed.

In another embodiment of the present disclosure, there can be provided a polymerizable composition comprising a polyisocyanate together with an ester thiol compound produced according to the above method, which further comprises other polythiol or polyol.

In yet another embodiment of the present disclosure, there can be provided a poly(thiol)urethane composition obtained by polymerizing the polythiol or polyol of the polymerizable composition and a reactive group of the polyisocyanate, wherein a molar ratio of SH (or OH) group/NCO group is in the range of 0.5 to 1.5.

In a further embodiment of the present disclosure, there can be provided a resin obtained by curing the poly(thiol) urethane composition, wherein the obtained resin may be used as an optical material, particularly an optical lens.

### Advantages of Invention

The production method of the present disclosure can improve the production efficiency and quality of ester thiol compounds, which are widely used in the chemical, resin, and coating industries, including the field of optical materials, and has high technical and industrial value.

The ester thiol compound obtained by the production method using a heterogeneous catalyst, which is a feature of the present disclosure has excellent economic efficiency and also can produce high-quality products. Additionally, by using this catalyst, high-quality optical resins and optical products can be obtained with high economic efficiency.

### Embodiments

The specific numerical values in blending ratio (content ratio), property value, and parameter used in the following description can be replaced with upper limit values (numerical values defined with “or less” or “below”) or lower limit values (numerical values defined with “or more” or “above”) of corresponding numerical values in blending ratio (content ratio), property value, and parameter described herein. The “parts” and “%” are based on mass unless otherwise specified.

Hereinafter, the method for producing an ester thiol compound of the present disclosure will be described in detail.

First, the present disclosure uses a heterogeneous catalyst as an ester catalyst used in the process of producing the ester thiol compound, so that the ester thiol compound of high purity can be easily produced without going through further complicated purification process.

As described above, the ester thiol compound is produced through an esterification method in which a polyhydric alcohol is reacted with a mercaptocarboxylic acid in the presence of an ester catalyst, while removing water, which is produced as a by-product. Esterification reaction catalysts generally include various catalysts such as Brønsted acids, Lewis acids or solid acid catalysts. A homogeneous catalyst was mainly used in a conventional ester reaction. Homogeneous catalysts that act in a molecular state have high activity, and react in a molecular state, and thus, they have excellent selectivity and are very effective. However, since the catalyst exists as one phase such as the reaction system, it is difficult to separate and reuse the product after completion of the reaction, and additional facility operations related to the separation require a lot of cost. Due to the chemicals and costs required for the separation process, it may deteriorate the economy of the process and even cause environmental pollution. Considering these points, the present disclosure uses a heterogeneous solid catalyst that is easy to separate and reuse. In particular, solid acid catalysts that have been developed along with the petrochemical industry are diverse and have a wide range of acid strength control, making them highly applicable to the production process of the present disclosure. Furthermore, if selectivity can be enhanced by using the pore structure of the solid catalyst, it is very useful in terms of suppressing the production of by-products and preventing environmental pollution.

A heterogeneous catalyst which can be used for the production of the ester thiol compound is not particularly limited, but a typical example thereof includes a natural clay, synthetic silica-alumina, zeolite, ion exchange resin, heteropoly acid, superstrong acid mesoporous material catalyst, or the like. Amorphous catalysts may also be used, but zeolite and mesoporous materials with regular pores can be used. In the catalyst whose pore size and shape are constant, shape-selective catalysis caused by the pore structure can also be expected. Zeolite pores are similar in size to ordinary molecules such as benzene, and when zeolite is used, materials can be distinguished according to the size of the molecule. By introducing such properties into the catalytic reaction, it is possible to adjust the reaction rate and improve the selectivity for a specific product by using the size of the reactants, the diffusion rate of the product, and the like, which is thus effective. As the size of the pores affects the transition state, it is also possible to enhance the selectivity for a specific product.

The zeolite is an aluminosilicate having crystallinity, and about 170 types of zeolites with different skeletal structures may be used. More specifically, MFI, FAU, MOR, BEA, LTA, and CHA may be mentioned. The physical and chemical properties of zeolite and its usability as a catalyst are determined by the structure of the skeleton, the size of the pore entrance, the shape and size of the pores, the Si/Al molar ratio, the size of the particles, and the like. The structure of the skeleton is the basic element of zeolite, and the shape of the skeleton is determined by the type of secondary basic units that make up the crystal and the method of bonding them. In addition, the shape, size, and connection method of the pores are determined by the skeleton, and the size of the pore entrance is also determined. If there are many large pores, the surface that can come into contact with the reactant is wide and thus, the activity as a catalyst can increase, but the mechanical stability is lowered. The Si/Al molar ratio, which indicates the degree to which aluminum is contained in the skeleton, is related to the generation of acid sites, and is also a major factor determining the hydrothermal stability of zeolite. The size of the particles determines the distance that reactants and products migrate within the zeolite. When the particles are small, the outer surface becomes wider, which makes them highly active in catalytic reactions that must first proceed on the outer surface. In addition, the catalytic properties of zeolite, especially its acidity, can be adjusted widely by various methods such as cation exchange, exchange of skeletal elements, support of specific substances, and shielding of acid sites on the outer surface, which makes it a very useful catalytic material.

In addition to such functions, it is possible to produce a very effective catalyst by utilizing the fact that the reaction properties change depending on the shape and size of the pores. However, the size of the pore entrance of zeolite is smaller than 0.7 nm, and thus, large molecules cannot enter the pores. Therefore, it cannot be used as a catalyst in organic synthesis reactions of large molecules. The mesopores in the range of 3 to 10 nm are developed in the mesoporous material announced in 1992, thereby capable of overcoming such a limitation. Not only the pore size and shape can be widely adjusted depending on the mold material used, but also aluminum is introduced into the skeleton along with silicon, which makes it highly feasible as an acid catalyst. However, unlike zeolite, since the pore walls are amorphous, strong acid sites are not generated and the pores are very large for the reactants. Therefore, it is difficult to expect an increase in selectivity due to interaction between the pore walls and the reactants. Instead, mesopores of uniform size are regularly developed, so that the surface area is large, material transfer is fast, a specific functional group is fixed to the pore wall, and thus, excellent performance as a catalyst can be expected.

In particular, the catalyst can be used by introducing a superstrong acid stronger than sulfuric acid into the mesoporous material. As the superstrong acid, sulfonic acid (RSO₃H), trifluoromethanesulfonic acid (CF₃SO₃H), perchloric acid (HClO₄), fluoric acid (HF), a derivative of sulfuric acid such as chlorosulfuric acid (ClSO₃H), fluorosulfuric acid (FSO₃H), and the like may be used.

A preferable superstrong acid may include superstrong acid mesoporous materials that have fixed sulfonic acid groups (—SO₃H) as sulfonic acid and appear to be heterogeneous catalysts, but have active sites that act like homogeneous catalysts, and the catalyst can be formed by using the same. Especially, when the sulfonic acid group is hung on the surface by an organic bond string, it becomes an acid catalyst effective in the organic synthesis reaction. Because the number of fixed sulfonic acid groups and the number of acid sites and the acid strength are determined by the peripheral structure, a solid acid catalyst suitable for the purpose can be produced. When silane containing an alkoxy group along with an —SH functional group is reacted with the surface hydroxyl group of a mesoporous material made of silica, the —SH functional group can be fixed to the pore wall. By treating this with an appropriate oxidizing agent, an acid catalyst in which a sulfonic acid group is fixed is produced. In addition, since it is synthesized by adding an alkoxysilane or chlorosilane with a —SH functional group or a sulfide bond to the synthesis mother liquor of the mesoporous material, the acidic functional group can be fixed to the pore wall during the synthesis process. If the Nafion group having very strong acid strength is fixed, very strong acid sites can be fixed to the pore wall of the mesoporous material. Unlike zeolite, the solid acid catalyst thus produced is difficult to expect shape selectivity due to its pore structure, but it has a wide surface area and fast material transfer, and is highly active in catalytic reactions of large molecules.

The polyhydric alcohol of Formula 1 used in the method for producing the ester thiol of the present disclosure is a commercially available substance and is not particularly limited, and examples thereof include pentaerythritol and the like.

Examples of the mercaptocarbonic acid of Formula 2 include, but are not limited to, 4-mercaptobutyric acid, 3-mercaptobutyric acid, 3-mercaptopropionic acid, 2-mercaptopropionic acid, 2-mercaptoacetic acid, and the like. In the present embodiment, or from the viewpoint of the lens polymerization reaction, 3-mercaptopropionic acid and 2-mercaptoacetic acid are preferred.

The weight ratio of the polyhydric alcohol of Formula 1 to the mercaptocarbonic acid of Formula 2 is 10˜40:90˜60. A preferred weight ratio is 20˜30:80˜70.

Meanwhile, the reactor used in the method for producing the ester thiol of the present disclosure may include a batch reactor, a tubular reactors, and the like. Among the tubular reactor, it is preferable to use a fixed bed tubular reactor that can be packed with the heterogeneous solid acid catalyst of the present disclosure. In particular, such a catalyst fixed bed reactor can be used by connecting multiple catalyst fixed beds depending on the purpose, and has the advantage of a high conversion rate per catalyst weight for the reaction. In addition, such a reactor has various advantages, such as easy production of products that are difficult to handle in a batch reactor and easy mass production because continuous production is possible.

The temperature of the process of producing the ester thiol compound according to the present disclosure is not particularly limited, but can be carried out at 0° C.˜250° C., preferably 50˜200° C., and more preferably 100˜180° C.

In order to eliminate oxygen in the production process of the present disclosure, it is preferable to perform it in a nitrogen atmosphere and a hydrogen atmosphere, and it can be carried out under a solvent or in a state where the solvent is removed.

The solvent used in the present disclosure is not particularly limited. The inactivated solvent may include aromatic hydrocarbons such as benzene, toluene, and xylene, for example, aliphatic hydrocarbons such as octane and decane, for example, alicyclic hydrocarbons such as cyclohexane, methylcyclohexane, and ethylcyclohexane, for example, halogenated aromatic hydrocarbons such as chlorotoluene, chlorobenzene, dichlorobenzene, dibromobenzene, and trichlorobenzene, for example, nitrogen-containing compounds such as nitrobenzene, N,N-dimethylformamide, N,N-dimethylacetateamide, and N,N′-dimethylimidazolidinone, for example, ethers such as dibutyl ether, ethylene glycol dimethyl ether, and ethylene glycol diethyl ether, for example, fatty acid esters such as amyl formate, n-butyl acetate, isobutyl acetate, n-amyl acetate, isoamyl acetate, methylisoamyl acetate, methoxybutyl acetate, 2-ethoxyethyl acetate, sec-hexyl acetate, 2-ethylbutyl acetate, 2-ethylhexyl acetate, cyclohexyl acetate, methylcyclohexyl acetate, benzyl acetate, ethyl propionate, n-butyl propynate, isoamyl propynate, ethyl acetate, butyl stearate, butyl lactate and amyl lactate, aromatic carboxylic acid esters such as methyl salicylate, dimethyl phthalate, and methyl benzoate, and the like, and can be used alone or in combination of two or more types. Preferable examples include aromatic hydrocarbons, and more preferable examples include toluene, chlorobenzene, and dichlorobenzene.

A product produced using the ester thiol compound obtained as described above can satisfy high optical properties and thus, they can be used in the production of optical materials, specifically plastic optical lenses.

According to the present disclosure, a polymerizable composition comprising a polythiol/polyol composition containing the previously described ester thiol compound, and an isocyanate composition is provided.

The polymerizable composition may include the isocyanate composition and polyol/polythiol in a mixed state or in a separated state. That is, in the polymerizable composition, the isocyanate composition and polyol/polythiol may be in a state in which they are blended contact with each other, or in a state in which they are separated so as not to come into contact with each other.

As the polyol components used in the polymerizable composition, for example, a low-molecular weight polyol and a high molecular weight polyol may be mentioned. The polyol may be used alone or in a mixture of two or more types.

The low-molecular weight polyol is a compound having two or more hydroxyl groups and having a number average molecular weight of 60 or more and less than 400. The low-molecular weight polyol may include, for example, dihydric alcohols such asethylene glycol, propylene glycol, 1,3-propenediol, 1,4-butylene glycol, 1,3-butylene glycol, 1,2-butylene glycol, 1,5-pentenediol, 1,6-hexenediol, diethylene glycol, triethylene glycol, dipropylene glycol, and mixtures thereof, 1,4-cyclohexenediol, hydrogenated bisphenol A, and bisphenol A, for example, trihydric alcohols such as glycerin, for example, tetrahydric alcohols such as tetramethylolmethane (pentaerythritol), for example, pentahydric alcohols such as xylitol, hexahydric alcohols such as sorbitol, mannitol, allitol, and iditol.

The high-molecular weight polyol is a compound having two or more hydroxyl groups and having a number average molecular weight of 400 or more, for example, 10000 or less, preferably 2000 or less. The high-molecular weight polyol may include, for example, polyetherpolyol, polyesterpolyol, polycarbonatepolyol, polyurethane polyol, epoxypolyol, vegetable oil polyol, polyolefinpolyol, acrylic polyol, silicone polyol, fluorine polyol, and vinyl monomer-modified polyol.

A polythiol component used in the polymerizable composition of the present disclosure including the ester thiol may include, for example, aliphatic polythiol, aromatic polythiol, heterocyclic-containing polythiol, an aliphatic polythiol containing a sulfur atom in addition to a mercapto group, an aromatic polythiol containing a sulfur atom in addition to a mercapto group, a heterocyclic-containing polythiol containing a sulfur atom in addition to a mercapto group, and the like. The thiol may be a thiol oligomer or polythiol, and may be used alone or in a mixture of two or more types. Specific examples of the thiol include 3,3′-thiobis[2-[(2-mercaptoethyl)thio]-1-propanethiol], bis(2-(2-mercaptoethylthio)-3-mercaptopropyl)sulfide, 4-mercaptomethyl-1,8-dimercapto-3,6-dithiaoctane, 2,3-bis(2-mercaptoethylthio)propane-1-thiol (GST), 2,2-bis(mercaptomethyl)-1,3-propanedithiol, bis(2-mercaptoethyl)sulfide, tetrakis(mercaptomethyl)methane, 2-(2-mercaptoethylthio) propane-1,3-dithiol, 2-(2,3-bis(2-mercaptoethylthio)propylthio)ethanethiol, bis(2,3-dimercaptopropanyl)sulfide, bis(2,3-dimercaptopropanyl)disulfide, 1,2-bis[(2-mercaptoethyl)thio]-3-mercaptopropane, 1,2-bis(2-(2-mercaptoethylthio)-3-mercaptopropylthio)ethane, 2-(2-mercaptoethylthio)-3-2-mercapto-3-[3-mercapto-2-(2-mercaptoethylthio)-propylthio]propylthio-propane-1-thiol, 2,2-bis-(3-mercapto-propionyloxymethyl)-butyl ester, 2-(2-mercaptoethylthio)-3-(2-(2-[3-mercapto-2-(2-mercaptoethylthio)-propylthio]ethylthio)ethylthio)propane-1-thiol, (4R,11S)-4,11-bis(mercaptomethyl)-3,6,9,12-tetrathiatetradecane-1,14-dithiol, (S)-3-((R-2,3-dimercaptopropyl)thio)propane-1,2-dithiol, (4R,14R)-4,14-bis(mercaptomethyl)-3,6,9,12,15-pentathiaheptane-1,17-dithiol, (S)-3-((R-3-mercapto-2-((2-mercaptoethyl)thio)propyl)thio)propyl)thio)-2-((2-mercaptoethyl)thio)propane-1-thiol, 3,3′-dithiobis(propane-1,2-dithiol), (7R,11S)-7,11-bis(mercaptomethyl)-3,6,9,12,15-pentathiaheptadecane-1,17-dithiol, (7R,12S)-7,12-bis(mercaptomethyl)-3,6,9,10,13,16-hexathioctadecane-1,18-dithiol, 5,7-dimercaptomethyl-1,11-dimercapto-3,6,9-trithiaundecane, 4,7-dimercaptomethyl-1,11-dimercapto-3,6,9-trithioundecane, 4,8-dimercaptomethyl-1,11-dimercapto-3,6,9-trithiaundecane, pentaerythritol tetrakis(3-mercaptopropionate), trimethylolpropane tris(3-mercaptopropionate), pentaethritol tetrakis(2-mercaptoacetate), bispentaerythritol ether hexakis(3-mercaptopropionate), 1,1,3,3-tetrakis(mercaptomethylthio)propane, 1,1,2,2-tetrakis(mercaptomethylthio)ethane, 4,6-bis(mercaptomethylthio)-1,3-dithiane, pentaerythritoltetrakis(2-mercaptoacetate), pentaerythritoltetrakis(3-mercaptopropionate) (PETMP), 2-(2,2-bis(mercaptodimethylthio)ethyl)-1,3-dithiane, and the like.

As an isocyanate composition used in the polymerizable composition of the present disclosure, at least one selected from an alkylene diisocyanate compound, an alicyclic diisocyanate compound, a heterocyclic diisocyanate compound, an aliphatic diisocyanate compound containing sulfur may be used.

Examples of the alkylene diisocyanate compound include ethylene diisocyanate; trimethylene diisocyanate; tetramethylene diisocyanate; 1,6-hexamethylene diisocyanate; octamethylene diisocyanate; nonamethylene diisocyanate; 2,2-dimethylpentane diisocyanate; 2,2,4-trimethylhexane diisocyanate; decamethylene diisocyanate; butene diisocyanate; 1,3-butadiene-1,4-diisocyanate; 2,4,4-trimethylhexamethylene diisocyanate; 1,6,11-undecane triisocyanate; 1,3,6-hexamethylene triisocyanate; 1,8-diisocyanato-4-isocyanatomethyloctane; 2,5,7-trimethyl-1,8-diisocyanato-5-isocyanatomethyloctane; bis(isocyanatoethyl)carbonate; bis(isocyanatoethyl)ether; 1,4-butylene glycol dipropyl ether-1,2-diisocyanate; 1,4-butylene glycol dipropyl ether-1,3-diisocyanate; 1,4-butylene glycol dipropyl ether-1,4-diisocyanate; 1,4-butylene glycol dipropyl ether-2,3-diisocyanate; 1,4-butylene glycol dipropyl ether-2,4-diisocyanate; methyl lysine diisocyanate; lysine triisocyanate; 2-isocyanatoethyl-2,6-diisocyanatohexanoate; 2-isocyanatopropyl-2,6-diisocyanatohexanoate; mesitylene triisocyanate; 2,6-di(isocyanatomethyl)furan, and the like.

Examples of the alicyclic diisocyanate compound include isophorone diisocyanate; dicyclohexylmethane diisocyanate; 3,8-bis(isocyanatomethyl)tricyclo[5,2,1,0,6]decane; 3,9-bis(isocyanatomethyl)tricyclo[5,2,1,0,6]decane; 4,8-bis(isocyanatomethyl)tricyclo[5,2,1,0,6]decane; 4,9-bis(isocyanatomethyl)tricyclo[5,2,1,0,6]decane; 2,5-bis(isocyanatomethyl)bicyclo[2,2,1]heptane; 2,6-bis(isocyanatomethyl)bicyclo[2,2,1]heptane; bis(isocyanatomethyl)cyclohexane; dicyclohexylmethane diisocyanate; cyclohexane diisocyanate; methylcyclohexanediisocyanate (DIMC); dicyclohexyldimethylmethane diisocyanate; 2,2′-dimethyldicyclohexylmethane diisocyanate; bis(4-isocyanato-n-butylidene)pentaerythritol; dimer acid diisocyanate; 2-isocyanatomethyl-3-(3-isocyanatopropyl)-5-isocyanatomethylbicyclo[2,2,1]-heptane; 2-isocyanatomethyl-3-(3-isocyanatopropyl)-6-isocyanatomethylbicyclo[2,2,1]-heptane; 2-isocyanatomethyl-2-(3-isocyanatopropyl)-5-isocyanatomethyl-bicyclo[2,2,1]-heptane; 2-isocyanatomethyl-2-(3-isocyanatopropyl)-6-isocyanatomethyl-bicyclo[2,2,1]-heptane; 2-isocyanatomethyl-3-(3-isocyanatopropyl)-6-(2-isocyanatoethyl)-bicyclo[2,2,1]-heptane; 2-isocyanatomethyl-3-(3-isocyanatopropyl)-6-(2-isocyanatoethyl)-bicyclo[2,2,1]-heptane; 2-isocyanatomethyl-2-(3-isocyanatopropyl)-5-(2-isocyanatoethyl)-bicyclo[2,2,1]-heptane; 2-isocyanatomethyl-2-(3-isocyanatopropyl)-6-(2-isocyanatoethyl)-bicyclo[2,2,1]-heptane; 1,3,5-tris(isocyanatomethyl)-cyclohexane; dicyclohexylmethane-4,4-diisocyanate (H12MDI), and the like.

Examples of the heterocyclic diisocyanate compound include thiophene-2,5-diisocyanate; methyl thiophene-2,5-diisocyanate; 1,4-dithiane-2,5-diisocyanate; methyl 1,4-dithiane-2,5-diisocyanate; 1,3-dithiolane-4,5-diisocyanate; methyl 1,3-dithiolane-4,5-diisocyanate; methyl 1,3-dithiolane-2-methyl-4,5-diisocyanate; ethyl 1,3-dithiolane-2,2-diisocyanate; tetrahydrothiophene-2,5-diisocyanate; methyltetrahydrothiophene-2,5-diisocyanate; ethyl tetrahydrothiophene-2,5-diisocyanate; methyl tetrahydrothiophene-3,4-diisocyanate; 1,2-diisothiocyanatoethane; 1,3-diisothiocyanatopropane; 1,4-diisothiocyanatobutane; 1,6-diisothiocyanatohexane; p-phenylenediisopropylidenediisothiocyanate; cyclohexane diisothiocyanate, and the like.

Examples of the aliphatic diisocyanate compound containing sulfur include 4-isocyanato-4′-isothiocyanatodiphenyl sulfide; 2-isocyanato-2′-isothiocyanatodiethyl disulfide; thiodiethyl diisocyanate; thiodipropyl diisocyanate; thiodihexyldiisocyanate; dimethylsulfone diisocyanate; dithiodimethyl diisocyanate; dithiodiethyl diisocyanate; dithiodipropyl diisocyanate; dicyclohexylsulfur-4,4′-diisocyanate; 1-isocyanatomethylthia-2,3-bis(2-isocyanatoethylthia)propane, and the like.

The polymerizable composition may further include additives such as an internal mold release agent, an ultraviolet absorber, a near-infrared absorber, a polymerization catalyst, a heat stabilizer, a color correcting agent, a chain extender, a crosslinking agent, a light stabilizer, an antioxidant, and a filler, if necessary.

As the internal mold release agent, components selected among a fluorine-based nonionic surfactant having a perfluoroalkyl group, a hydroxyalkyl group, or a phosphoric acid ester group; a silicone-based nonionic surfactant having a dimethylpolysiloxane group, a hydroxyalkyl group, or a phosphoric acid ester group; an alkyl quaternary ammonium salt, that is, a trimethylcetyl ammonium salt, a trimethylstearyl, dimethylethylcetyl ammonium salt, a triethyldodecyl ammonium salt, a trioctylmethyl ammonium salt, a diethylcyclohexadodecyl ammonium salt; and an acidic phosphate ester may be used alone or in combination of two or more types.

As the ultraviolet absorber, benzophenone-based, benzotriazole-based, triazine-based, salicylate-based, cyanoacrylate-based, oxanilide-based, and the like may be used.

As the near-infrared absorber, azo-based, amino-based, anthraquinone-based, cyanine-based, polymethine-based, diphenylmethane-based, triphenylmethane-based, quinone-based, diammonium-based, dithiol metal complex-based, squarylium-based, phthalocyanine-based, naphthalocyanine-based etc. may be used.

As the polymerization catalyst, amine-based, phosphorus-based, organotin-based, organo-copper-based, organogallium, organozirconium, organo-iron-based, organozinc, organoaluminum, organobismuth-based, and the like may be used. Specifically, a tin compounds such as dibutyltin dilaurate, dibutyltin dichloride, dimethyltin dichloride, tetramethyldiacetoxydistanoxane, tetraethyldiacetoxydistanoxane, tetrapropyldiacetoxydistanoxane, tetrabutyldiacetoxydistanoxane, or an amine compound such as tertiary amine may be used. These can be used alone or in combination of two or more types. The amount of the catalyst added is preferably in the range of 0.001 to 1% by weight based of the total weight of the monomer of the composition. In the case of this range, it is preferable not only in terms of polymerizability, but also in terms of pot life, transparency of the resulting resin, various optical properties, and light resistance.

As the heat stabilizer, metal fatty acid salt-based, phosphorus-based, lead-based, organotin-based, and the like may be used alone or in a mixture of two or more types.

Further, the resin composition for the optical lens can further contain a color correcting agent for correcting the initial color of the lens. As the color correcting agent, an organic dye, an organic pigment, an inorganic pigment, and the like may be used. By adding the organic dye or the like in an amount of 0.1˜50,000 ppm, preferably 0.5˜10,000 ppm, per resin composition for the optical lens, it is possible to prevent the lens from turning yellow due to the addition of an ultraviolet absorber, optical resin, monomer, and the like.

Further, according to the present disclosure, a poly(thio) urethane obtained from the above-mentioned polymerizable composition is provided. That is, the poly(thio)urethane may be produced by polymerizing (and curing) the isocyanate composition and thiol in the polymerizable composition. In the polymerization reaction, the molar ratio of the SH group/NCO group may be 0.5˜1.5 or more preferably 0.9˜1.1.

When curing the composition, there may be various molding methods depending on the application, and although the curing method is not particularly limited, generally curing by heat is mainly used. Consequently, the resin of the present disclosure is obtained. The resin of the present disclosure is obtained by casting polymerization which is a conventional mold injection method.

The process for producing a spectacle lens by heat curing the resin composition of the present disclosure is as follows. First, a polymerization initiator is finally added to the composition, then nitrogen is blown to remove air into the mixing cylinder, then decompression and stirring are carried out for 1˜5 hours, the stirring is stopped, then decompression and defoaming are carried out, and the mixture is injected into the mold. At this time, the mold used is preferably a plastic gasket or a glass mold or a metal mold fixed with a polyester or polypropylene adhesive tape. A glass mold injected with the mixture is put into a forced circulation oven, and the temperature is slowly raised from room temperature to 120˜130° C., maintained at 120˜140° C. for 1˜4 hours, cooled slowly to 60˜80° C., and then the solid is released from the mold to obtain an optical lens. The optical lens thus obtained is annealed at a temperature of 120˜140° C. for 1˜ 4 hours to obtain the final desired plastic eyeglass lens (fabric).

Further, the optical lens obtained by the above method can be subjected to hard coating and multi-coating treatment to enhance optical properties. In the formation of the hard coating layer, a coating composition mainly composed of at least one silane compound having a functional group such as an epoxy group, an alkoxy group, or a vinyl group, and at least one metal oxide colloid such as silicic acid oxide, titanium oxide, antimony oxide, tin oxide, tungsten oxide, and aluminum oxide is coated onto the surface of the optical lens to a thickness of 0.5˜10 by impregnation or spin coating, and then heated or cured with ultraviolet rays to complete the coating film.

The multi-coating layer, that is, the anti-reflection coating layer, can be formed by vacuum deposition or sputtering of metal oxide such as silicon oxide, magnesium fluoride, aluminum oxide, zirconium oxide, titanium oxide, tantalum oxide, and yttrium oxide. Most preferably, silicon oxide and zirconium oxide films are repeatedly vacuum-deposited three or more times on the hard coating film on both sides of the lens, and then the silicon oxide film is finally vacuum-deposited. Additionally, if necessary, a water film (fluorine resin) layer can be placed at the end, or an ITO layer can be placed between the silicon oxide and zirconium oxide films.

The optical lens of the present disclosure may be used after coloring using a disperse dye or a light discoloration dye, if necessary.

The resin composition for the optical lens is not limited to a plastic spectacle lens, and can be used for various optical products.

It is necessary to evaluate whether the optical lens made by the present disclosure has appropriate physical properties as a plastic spectacle lens, and respective property values, (1) refractive index and Abbe number (υ), (2) heat resistance (Tg), (3) yellowness, (4) striae and white turbidity, were evaluated by the following test method.

(1) Refractive index (nE20): The refractive index of 546 nm (E wavelength) was measured at 20° C. using an ABBE refractometer, DR-M4 model from ATAGO.
(2) Heat resistance: The glass transition temperature (Tg) of the test specimen was measured using SCINCO's DSC N-650 heat analyzer to determine heat resistance.
(3) Yellowness: The Y.I value of a 2 mm test specimen was measured using SHIMADZU's UV-2600 spectrometer to compare the yellowness.
(4) striae and white turbidity: 100 lenses are observed with the naked eye, and the degree of occurrence of striae and whitening are marked as 10 or more: X, 5˜9: Δ, 3˜4: ◯, and 2 or less: ⊚.

The present disclosure is illustrated with reference to the following examples, but is not limited thereby.

Abbreviations used in the examples mean:
TESPT: Bis(tetraethoxysilyl)tetrasulfide
TEOS: Tetraethyl orthosilicate
CTABr: Cetyltrimethylammonium bromide
PETMA: Pentaerythritol tetrathioglycolic acid
TPA: dodecatungstophosphoric acid

### 1. Preparation of Superacid Mesoporous Material Catalyst

### Synthesis Example 1-1

In a 300 ml Teflon reactor, 5 g of CTABr (0.0137 mol) was dissolved in a solution containing 100 ml of deionized water and 50 ml of 28% ammonium hydroxide (0.55 mol), and then stirred for 15 minutes. 9 g of TEOS (0.038 mol) and 2.59 g TESPT (0.0096 mol) were further added for 30 minutes, and stirred for 2 hours. The Teflon reactor was then put into an autoclave reactor, and aged at 100° C. for 3 days. Then, the solid solution was filtered and thoroughly washed with deionized water and ethanol. The obtained solid was dried in an oven at 100° C. and calcined at 500° C.

1 g of the obtained solid was dispersed in 50 ml of 35% hydrochloric acid, then 2 g of bromine (Br₂) was added at −10° C. for 1 hour and stirred at room temperature for 10 hours. The resulting solid was washed with 100 ml of purified water, washed again with 200 ml of methanol, and the dried under vacuum to obtain a superacid mesoporous material (TESPT-10-MCM) into which sulfonic acid was introduced.

### Synthesis Examples 1-2 to 1-3

In the same manner as in Synthesis Example 1-1, superacid mesoporous materials, TESPT-20-MCM and TESPT-30-MCM, were obtained according to the compositions shown in Table 1 below.

TABLE 1

| Synthesis Example [symbol] | TEOS (g) [symbol] | TESPT (g) [symbol] | Remarks [symbol] |
|---|---|---|---|
| 1-1 [symbol] | 9 [symbol] | 2.59 [symbol] | TESPT-10-MCM [symbol] |
| 1-2 [symbol] | 8 [symbol] | 5.18 [symbol] | TESPT-20-MCM [symbol] |
| 1-3 [symbol] | 7 [symbol] | 7.7 [symbol] | TESPT-30-MCM [symbol] |

### 2. Preparation of Ester Thiol Compound

### Synthesis Example 2-1

100 g of pentaerythritol (Samyang Chemical, 99%), 5 g of superstrong acid mesoporous material (TESPT-10-MCM) obtained in Synthesis Example 1-1, 300 ml of toluene and 312 g of 3-mercaptopropionic acid (Alpha A, 99%) were put into a reactor equipped with a Dean-Stock apparatus, a nitrogen gas purge tube, and a thermometer, and the internal temperature of the reactor was heated to the toluene reflux temperature. The reaction proceeded for 4 hours while continuously removing the produced water, and then cooled to room temperature. The amount of water removed was 99.0% relative to the theoretically calculated amount of water.

The reaction solution was cooled to room temperature, filtered to separate the catalyst, and then concentrated under reduced pressure to obtain 330 g of pentaerythritol tetrakis(3-mercaptopropionate) (hereinafter abbreviated as PETMP) which is an ester thiol compound. The obtained PETMP was confirmed to be pentaerythritol tetrakis (3-mercaptopropionate) through high-performance liquid chromatography and infrared spectroscopy, and the APHA was 6.

### Synthesis Examples 2-2 to 2-6, Comparative Synthesis Example 1

PETMP was obtained in the same manner as in Synthesis Example 2-1, except that the catalyst listed in Table 2 was used instead of the superstrong acid mesoporous material (TESPT-10-MCM). The obtained PETMP was confirmed through high-performance liquid chromatography and infrared spectroscopy as in Synthesis Example 2-1, and the results are listed in Table 2 below.

### Synthesis Examples 2-7 to 2-8

PETMP was obtained in the same manner as in Synthesis Example 1, except that the catalyst used in Synthesis Examples 2-2 and 2-6 was reused as a catalyst. The obtained PETMP results were similar to those using a novel catalyst.

### Synthesis Example 2-9

100 g of pentaerythritol, 5 g of superstrong acid mesoporous material (TESPT-20-MCM) obtained in Synthesis Example 1-2, 300 ml of toluene and 271 g of mercaptoacetic acid were put into a reactor equipped with a Dean-Stock apparatus, a nitrogen gas purge tube, and a thermometer, and the internal temperature of the reactor was heated to the toluene reflux temperature. The reaction proceeded for 4 hours while continuously removing the produced water, and then cooled to room temperature. The reaction solution was cooled to room temperature, filtered to separate the catalyst, and then concentrated under reduced pressure to obtain 312 g of pentaerythritol tetrakis(3-mercaptoacetate) (hereinafter abbreviated as PETMA) which is an ester thiol compound. The obtained PETMA was confirmed through high-performance liquid chromatography and infrared spectroscopy as in Synthesis Example 2-1, and the APHA was 7.

### Synthesis Comparative Example 1

100 g of pentaerythritol (Samyang Chemical, 99%), 5 g of methanesulfonic acid, 300 ml of toluene, and 312 g of thioglycolic acid (Dicel, 99%) were put into a reactor equipped with a Dean-Stock apparatus, a nitrogen gas purge tube, and a thermometer, and the internal temperature of the reactor was heated to the toluene reflux temperature. The reaction proceeded for 4 hours while continuously removing the produced water, and then cooled to room temperature. Then, 246 g of 5% aqueous sodium carbonate solution was added to the obtained reaction product, and stirred at 25° C. for 1 hour. The layers were separated to remove the lower layer, and further washed twice with 246 g of distilled water. Then, toluene and trace amounts of water were completely by heating and concentrating under vacuum, and then filtered to obtain 337 g of PETMP which is an ester thiol compound. The color of the obtained ester thiol compound was APHA 15.

TABLE 2

| | | | | Amount of |
|---|---|---|---|---|
| | | | | wastewater |
| Example [symbol] | Catalyst [symbol] | Yield [symbol] | APHA [symbol] | generated [symbol] |
| Synthesis | TESPT-10-MCM [symbol] | 97% [symbol] | 6 [symbol] | 60 ml [symbol] |
| Example 2-1 [symbol] | | | | |
| Synthesis | TESPT-20-MCM [symbol] | 99% [symbol] | 5 [symbol] | 60 ml [symbol] |
| Example 2-2 [symbol] | | | | |
| Synthesis | TESPT-30-MCM [symbol] | 98% [symbol] | 5 [symbol] | 60 ml [symbol] |
| Example 2-3 [symbol] | | | | |
| Synthesis | T P A [symbol] | 98% [symbol] | 9 [symbol] | 60 ml [symbol] |
| Example 2-4 [symbol] | | | | |
| Synthesis | Zeolite (ZSM-5) [symbol] | 96% [symbol] | 6 [symbol] | 60 ml [symbol] |
| Example 2-5 [symbol] | | | | |
| Synthesis | Y Zeolite [symbol] | 98% [symbol] | 6 [symbol] | 60 ml [symbol] |
| Example 2-6 [symbol] | | | | |
| Synthesis | TESPT-20-MCM | 99% [symbol] | 6 [symbol] | 60 ml [symbol] |
| Example 2-7 [symbol] | (reused) [symbol] | | | |
| Synthesis | s Zeolite (reused) [symbol] | 97% [symbol] | 5 [symbol] | 60 ml [symbol] |
| Example 2-8 [symbol] | | | | |
| Synthesis | TESPT-20-MCM [symbol] | 98% [symbol] | 7 [symbol] | 30 ml [symbol] |
| Example 2-9 [symbol] | | | | |
| Synthesis | Methysulfonic | 94% [symbol] | 15 [symbol] | 820 ml [symbol] |
| Comparative | acid [symbol] | | | |
| Example 1 [symbol] | | | | |

An ester thiol compound excellent in yield and color can be synthesized by using a heterogeneous solid acid catalyst for the production of the ester thiol compound, and in particular, the amount of wastewater generated is significantly smaller than that of Synthesis Comparative Example 1 using the homogeneous catalyst. Further, as shown in Synthesis Examples 2-7 and 2-8, it was possible to reuse the heterogeneous solid acid catalyst in the production process of the present disclosure. Thereby, through the introduction of a heterogeneous solid acid catalyst in the production of the ester thiol compound, it will be possible to apply a continuous production method through a catalyst layer from the existing batch production method.

### 3. Production and Evaluation of Optical Lens

### Example 1

30.8 g of 2,3-bis(2-mercaptoethylthio)propane-1-thiol (GST), 19.7 g of pentaerythritol tetrakis(3-mercaptopropionate) (PETMP) prepared in the above Synthesis Example, 0.1 g of dibutyltin dichloride (DBDC), 0.12 g of ZELEC UN, and 1.5 g of 2-(2-hydroxy-5-t-octylphenyl)-2H-benzotriazole were added to 46.5 g of methylcyclohexane diisocyanate (DIMC), and the mixture was stirred for 10 minutes under a nitrogen stream, vacuum degassed at less than 1 torr for 1 hour, purged with nitrogen, and injected into a glass mold using nitrogen pressure. The glass mold into which the optical resin composition was injected was slowly heated from 30° C. to 125° C. in a forced circulation oven, maintained at 125° C. for 2 hours, then cooled to 70° C., and the optical resin was demolded from the mold to obtain a spectacle lens with a central thickness of 1.3 mm.

The obtained spectacle lens was processed to a width of 72 mm, then immersed in an aqueous alkaline cleaning solution, ultrasonically cleaned, and then annealed at 125° C. for 2 hours. The hard liquid was then dip-coated, heat cured, and then metal oxide and fluorine resin were vacuum deposited to obtain multi-coated spectacle lenses.

### Examples 2 to 8, Comparative Example 1

An optical resin was produced in the same manner as in Example 1, except that PETMP listed in Table 3 was used, and the physical properties were evaluated. The results are listed in Table 3 below.

TABLE 3

| | | Refractive | Heat | | | White |
|---|---|---|---|---|---|---|
| [symbol] | PETMP [symbol] | index [symbol] | resistance [symbol] | Yellowness [symbol] | Striae [symbol] | turbidity [symbol] |
| Example 1 [symbol] | Synthesis Example 2-1 [symbol] | 1.5956 [symbol] | 139.86 [symbol] | 1.26 [symbol] | ⊚ [symbol] | ⊚ [symbol] |
| Example 2 [symbol] | Synthesis Example 2-2 [symbol] | 1.5961 [symbol] | 140.12 [symbol] | 1.25 [symbol] | ⊚ [symbol] | ⊚ [symbol] |
| Example 3 [symbol] | Synthesis Example 2-3 [symbol] | 1.5958 [symbol] | 139.98 [symbol] | 1.27 [symbol] | ⊚ [symbol] | ⊚ [symbol] |
| Example 4 [symbol] | Synthesis Example 2-4 [symbol] | 1.5951 [symbol] | 138.56 [symbol] | 1.46 [symbol] | ⊚ [symbol] | ◯ [symbol] |
| Example 5 [symbol] | Synthesis Example 2-5 [symbol] | 1.5957 [symbol] | 139.68 [symbol] | 1.30 [symbol] | ⊚ [symbol] | ◯ [symbol] |
| Example 6 [symbol] | Synthesis Example 2-6 [symbol] | 1.3953 [symbol] | 139.54 [symbol] | 1.28 [symbol] | ⊚ [symbol] | ◯ [symbol] |
| Example 7 [symbol] | Synthesis Example 2-7 [symbol] | 1.5963 [symbol] | 140.35 [symbol] | 1.26 [symbol] | ⊚ [symbol] | ⊚ [symbol] |
| Example 8 [symbol] | Synthesis Example 2-8 [symbol] | 1.5958 [symbol] | 139.52 [symbol] | 1.27 [symbol] | ⊚ [symbol] | ⊚ [symbol] |
| Comparative | Synthesis Comparative | 1.5951 [symbol] | 138.62 [symbol] | 1.82 [symbol] | ◯ [symbol] | ◯ [symbol] |
| Example 1 [symbol] | Example 1 [symbol] | | | | | |

Through the Examples, the ester thiol compound produced using a heterogeneous solid acid catalyst can be used as an optical lens. When comparing Example 2 and Comparative Example 1, the yellowness showed a very large difference of 1.25 and 1.82, and the lens using an ester thiol compound produced with a heterogeneous solid, showed very excellent results in terms of yellowness as compared to the lens produced with a homogeneous catalyst.

In addition, when comparing Examples and Comparative Examples, a lens using the ester thiol compound produced with a heterogeneous solid acid catalyst also showed good results in terms of optical properties such as striae and white turbidity as compared to the lens produced using a homogeneous catalyst, and thus could be used as an optical material.

1. A method for producing an ester thiol compound of Formula 3 below, the method comprising:
reacting a polyalcohol of Formula 1 below and a mercaptocarboxylic acid of Formula 2 below by using a catalyst,
wherein the catalyst is prepared by introducing superstrong acid into mesoporous materials with pore diameter ranging from 2 to 10 nm: 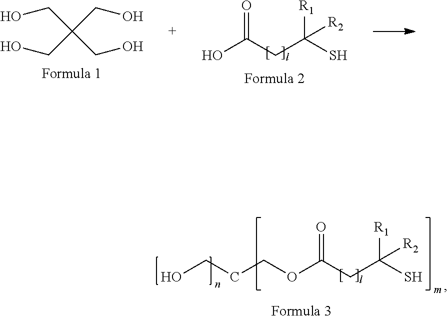
wherein, R₁ and R₂ are each independently H or CH₃, l is an integer of 0 to 2, m is an integer of 1 to 4, and n is an integer of 0 to 3.

2. The method according to claim 1,
wherein the superstrong acid is one or more substances selected from the group consisting of sulfonic acid (RSO₃H), trifluoromethanesulfonic acid (CF₃SO₃H), perchloric acid (HClO₄), fluoric acid (HF), and chlorosulfuric acid (ClSO₃H) and fluorosulfuric acid (FSO₃H) as derivative of sulfuric acid.

3. The method according to claim 1,
wherein the catalyst is a superstrong acid mesoporous material catalyst.

4. The method according to claim 2,
wherein the superstrong acid is sulfonic acid.

5. The method according to claim 1,
wherein the catalyst is used in a weight ratio of 0.2 to 20, based on 100 g of the polyhydric alcohol.

6. The method according to claim 1,
wherein the polyalcohol of Formula 1 and the mercaptocarboxylic acid of Formula 2 are used in a weight ratio of 20˜30 to 80˜70.

7. The method according to claim 1,
wherein the method for producing the ester thiol compound is carried out in a fixed bed tube type reactor.

8. A polymerizable poly(thio)urethane composition for optical materials, comprising:
(i) an ester thiol compound of the formula (3) below;
(ii) a polyisocyanate; and
(iii) a polythiol or a polyol, wherein the polythiol is different from the ester thiol compound represented by the formula (3) below: 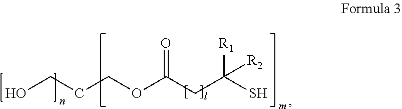
wherein, R₁ and R₂ are each independently H or CH₃, l is an integer of 0 to 2, m is an integer of 1 to 4, and n is an integer of 0 to 3.

9. The polymerizable poly(thiol)urethane composition according to claim 8,
wherein a molar ratio of [SH (or OH) group of the polythiol or polyol]/[NCO group of the polyisocyanate] is in the range of 0.5 to 1.5.

10. An optical lens comprising:
poly(thiol)urethane resin,
wherein the poly(thiol)urethane resin is a cured product of the polymerizable poly(thiol) urethane composition of claim 8.

11-12. (canceled)
